SEQUENCE LISTING (16 sequences)

SEQ ID NO: 1 - DNA, 53 bp, Artificial Sequence
  Vector AsiSI sense primer
cggggacgtggttttcctttgaaaaacgcgatcgcaccatggcgcctattacg

SEQ ID NO: 2 - DNA, 53 bp, Artificial Sequence
  Vector AsiSI anti-sense primer
cgtaataggcgccatggtgcgatcgcgtttttcaaaggaaaaccacgtccccg

SEQ ID NO: 3 - DNA, 40 bp, Artificial Sequence
  Vector FseI sense primer
gatgagatggaagggccggcctcacacctcccttacatcg

SEQ ID NO: 4 - DNA, 40 bp, Artificial Sequence
  Vector FseI anti-sense primer
cgatgtaagggaggtgtgaggccggcccttccatctcatc

SEQ ID NO: 5 - DNA, 10076 bp, Artificial Sequence
  pSC_1b_NS3/4A/lacZ_AsiSI_FseI
cctgcaggtaatacgactcactatagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcgcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaaggaaggcccggcgccattctatcctcttgaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggttatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacgcgatcgcaccgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgggccggcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccatt

SEQ ID NO: 6 - DNA, 38 bp, Artificial Sequence
  Vector FspAI sense primer
gtctccgggagctgggtgcgcatcacacctcccttaca

SEQ ID NO: 7 - DNA, 38 bp, Artificial Sequence
  Vector FspAI anti-sense primer
tgtaagggaggtgtgatgcgcacccagctcccggagac

SEQ ID NO: 8 - DNA, 10076 bp, Artificial Sequence
  pSC_1b_NS3/4A/lacZ_AsiSI_FspAI
cctgcaggtaatacgactcactatagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcgcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaaggaaggcccggcgccattctatcctcttgaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggttatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacgcgatcgcaccgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgggtgcgcatcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccatt

SEQ ID NO: 9 - DNA, 19 bp, Artificial Sequence
  Reverse transcription anti-sense primer
accaggtcctcsgtggagg

SEQ ID NO: 10 - DNA, 18 bp, Artificial Sequence
  1st round PCR sense primer
gagaccaagatcatcacc

SEQ ID NO: 11 - DNA, 16 bp, Artificial Sequence
  1st round PCR anti-sense primer
ccaccacgggagcagc

SEQ ID NO: 12 - DNA, 21 bp, Artificial sequence
  2nd round PCR sense primer
ggcagacaccgcggcgtgtgg

SEQ ID NO: 13 - DNA, 19 bp, Artificial Sequence
  2nd round PCR anti-sense primer
ttccacatgtgcttcgccc

SEQ ID NO: 14 - DNA, 44 bp, Artificial Sequence
  Sense primer AsiSI
ctgtctgtctgcgatcgcaccatggcgcctattacggcctactc

SEQ ID NO: 15 - DNA, 30 bp, Artificial Sequence
  Anti-sense primer FspAI
agggaggtgtgatgcgcactcttccatctc

SEQ ID NO: 16 - DNA, 25 bp, Artificial Sequence
  Anti-sense primer FseI
cgcactcttccatctcatcgaactc

(19) United States
(12) Patent Application Publication
(10) Pub. No.: US 2011/0053160 A1
(43) Pub. Date: Mar. 3, 2011

(54) HCV NS3/4A REPLICON SHUTTLE VECTORS

(76) Inventors: SAMIR ALI, NORTHRIDGE, CA (US); MILLONI B. CHHABRA, DURHAM, NC (US); MARIA JOLANTA ILNICKA, WEST CALDWELL, NJ (US); WEN-RONG JIANG, REDWOOD CITY, CA (US); ISABEL NAJERA, PORTOLA VALLEY, CA (US)

(21) Appl. No.: 12/872,124

(22) Filed: Aug. 31, 2010

Related U.S. Application Data
(60) Provisional application No. 61/238,429, filed on Aug. 31, 2009.

(51) Int. Cl. C12Q 1/68; C12N 15/86

(52) U.S. Cl. 435/6; 435/320.1

(57) ABSTRACT

The present invention provides for novel HCV NS3/4A replicon shuttle vectors useful for cloning in HCV polynucleotide sequences from samples of HCV-infected patients and testing the resulting replicons for drug susceptibility.

### CROSS REFERENCE TO RELATED INVENTION

This application claims the benefit of priority of U.S. Provisional Patent Application Ser. No. 61/238,429, filed Aug. 31, 2009, which is incorporated herein by reference in its entirety.

### REFERENCE TO SEQUENCE LISTING

This application contains a Sequence Listing submitted as an electronic text file named “Case_{—}22699_US_ST25.txt”, having a size in bytes of 29 kb, and created on 31 Aug., 2009. The information contained in this electronic file is hereby incorporated by reference in its entirety pursuant to 37 CFR §1.52(e)(5).

### FIELD OF THE INVENTION

This invention pertains to novel HCV NS3-4A replicon shuttle vectors which are useful for screening, testing and evaluating HCV protease and helicase inhibitors.

### BACKGROUND OF THE INVENTION

Hepatitis C virus is a major health problem and the leading cause of chronic liver disease throughout the world. (Boyer, N. et al. J. Hepatol. 2000 32:98-112). Patients infected with HCV are at risk of developing cirrhosis of the liver and subsequent hepatocellular carcinoma and hence HCV is the major indication for liver transplantation.

According to the World Health Organization, there are more than 200 million infected individuals worldwide, with at least 3 to 4 million people being infected each year. Once infected, about 20% of people clear the virus, but the rest can harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring. Current treatments for HCV infection, which are restricted to immunotherapy with recombinant interferon-α alone or in combination with the nucleoside analog ribavirin, are of limited clinical benefit particularly for genotype 1. There is an urgent need for improved therapeutic agents that effectively combat chronic HCV infection

HCV has been classified as a member of the virus family Flaviviridae that includes the genera flaviviruses, pestiviruses, and hepaciviruses which includes hepatitis C viruses (Rice, C. M., Flaviviridae: The viruses and their replication, in: Fields Virology, Editors: Fields, B. N., Knipe, D. M., and Howley, P. M., Lippincott-Raven Publishers, Philadelphia, Pa., Chapter 30, 931-959, 1996). HCV is an enveloped virus containing a positive-sense single-stranded RNA genome of approximately 9.4 kb. The viral genome consists of a 5′-untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation.

Genetic analysis of HCV has identified six main genotypes showing a >30% divergence in the DNA sequence. Each genotype contains a series of more closely related subtypes which show a 20-25% divergence in nucleotide sequences (Simmonds, P. 2004 J. Gen. Virol. 85:3173-88). More than 30 subtypes have been distinguished. In the US approximately 70% of infected individuals have type 1a and 1b infection. Type 1b is the most prevalent subtype in Asia. (X. Forms and J. Bukh, Clinics in Liver Disease 1999 3:693-716; J. Bukh et al., Semin. Liv. Dis. 1995 15:41-63). Unfortunately Type 1 infections are less responsive to the current therapy than either type 2 or 3 genotypes (N. N. Zein, Clin. Microbiol. Rev., 2000 13:223-235).

The genetic organization and polyprotein processing of the nonstructural protein portion of the ORF of pestiviruses and hepaciviruses is very similar. These positive stranded RNA viruses possess a single large open reading frame (ORF) encoding all the viral proteins necessary for virus replication. These proteins are expressed as a polyprotein that is co- and post-translationally processed by both cellular and virus-encoded proteinases to yield the mature viral proteins. The viral proteins responsible for the replication of the viral genome RNA are located towards the carboxy-terminal. Two-thirds of the ORF are termed nonstructural (NS) proteins. For both the pestiviruses and hepaciviruses, the mature nonstructural (NS) proteins, in sequential order from the amino-terminus of the nonstructural protein coding region to the carboxy-terminus of the ORF, consist of p7, NS2, NS3, NS4A, NS4B, NS5A, and NS5B.

The NS proteins of pestiviruses and hepaciviruses share sequence domains that are characteristic of specific protein functions. For example, the NS3 proteins of viruses in both groups possess amino acid sequence motifs characteristic of serine proteinases and of helicases (Gorbalenya et al. Nature 1988 333:22; Bazan and Fletterick Virology 1989 171:637-639; Gorbalenya et al. Nucleic Acid Res. 1989 17.3889-3897). Similarly, the NS5B proteins of pestiviruses and hepaciviruses have the motifs characteristic of RNA-directed RNA polymerases (Koonin, E. V. and Dolja, V. V. Crit. Rev. Biochem. Molec. Biol. 1993 28:375-430).

The actual roles and functions of the NS proteins of pestiviruses and hepaciviruses in the lifecycle of the viruses are directly analogous. In both cases, the NS3 serine proteinase is responsible for all proteolytic processing of polyprotein precursors downstream of its position in the ORF (Wiskerchen and Collett Virology 1991 184:341-350; Bartenschlager et al. J. Virol. 1993 67:3835-3844; Eckart et al. Biochem. Biophys. Res. Comm. 1993 192:399-406; Grakoui et al. J. Virol. 1993 67:2832-2843; Grakoui et al. Proc. Natl. Acad. Sci. USA 1993 90:10583-10587; Ilijikata et al. J. Virol. 1993 67:4665-4675; Tome et al. J. Virol. 1993 67:4017-4026). The NS4A protein, in both cases, acts as a cofactor with the NS3 serine protease (Bartenschlager et al. J. Virol. 1994 68:5045-5055; Fulla et al. J. Virol. 1994 68: 3753-3760; Xu et al. J. Virol. 1997 71:53 12-5322). The NS3 protein of both viruses also functions as a helicase (Kim et al. Biochem. Biophys. Res. Comm. 1995 215: 160-166; Jin and Peterson Arch. Biochem. Biophys. 1995, 323:47-53; Warrener and Collett J. Virol. 1995 69:1720-1726). Finally, the NS5B proteins of pestiviruses and hepaciviruses have the predicted RNA-dependent RNA polymerase activity (Behrens et al. EMBO 1996 15:12-22; Lechmann et al. J. Virol. 1997 71:8416-8428; Yuan et al. Biochem. Biophys. Res. Comm. 1997 232:231-235; Hagedorn, PCT WO 97/12033; Zhong et al. J. Virol. 1998 72:9365-9369).

Currently there are a limited number of approved therapies are currently available for the treatment of HCV infection. New and existing therapeutic approaches to treating HCV and inhibition of HCV NS5B polymerase have been reviewed: R. G. Gish, Sem. Liver. Dis., 1999 19:5; Di Besceglie, A. M. and Bacon, B. R., Scientific American, October: 1999 80-85; G. Lake-Bakaar, Current and Future Therapy for Chronic Hepatitis C Virus Liver Disease, Curr. Drug Targ. Infect Dis. 2003 3(3):247-253; P. Hoffmann et al., Recent patents on experimental therapy for hepatitis C virus infection (1999-2002), Exp. Opin. Ther. Patents 2003 13(11):1707-1723; F. F. Poordad et al. Developments in Hepatitis C therapy during 2000-2002, Exp. Opin. Emerging Drugs 2003 8(1):9-25; M. P. Walker et al., Promising Candidates for the treatment of chronic hepatitis C, Exp. Opin. Investig. Drugs 2003 12(8):1269-1280; S.-L. Tan et al., Hepatitis C Therapeutics: Current Status and Emerging Strategies, Nature Rev. Drug Discov. 2002 1:867-881; R. De Francesco et al. Approaching a new era for hepatitis C virus therapy: inhibitors of the NS3-4A serine protease and the NS5B RNA-dependent RNA polymerase, Antiviral Res. 2003 58:1-16; Q. M. Wang et al. Hepatitis C virus encoded proteins: targets for antiviral therapy, Drugs of the Future 2000 25(9):933-8-944; J. A. Wu and Z. Hong, Targeting NS5B-Dependent RNA Polymerase for Anti-HCV Chemotherapy Cur. Drug Targ.-Inf Dis 2003 3:207-219.

Despite advances in understanding the genomic organization of the virus and the functions of viral proteins, fundamental aspects of HCV replication and pathogenesis remain unknown. A major challenge in gaining experimental access to HCV replication is the lack of an efficient cell culture system that allows production of infectious virus particles. Although infection of primary cell cultures and certain human cell lines has been reported, the amounts of virus produced in those systems and the levels of HCV replication have been too low to permit detailed analyses.

The construction of selectable subgenomic HCV RNAs that replicate with minimal efficiency in the human hepatoma cell line Huh-7 has been reported. Lohman et al. reported the construction of a replicon (I₃₇₇/NS3-3′) derived from a cloned full-length HCV consensus genome (genotype 1b) by deleting the C-p7 or C—NS2 region of the protein-coding region (Lohman et al., Science 1999 285: 110-113). The replicon contained the following elements: (i) the HCV 5′-UTR fused to 12 amino acids of the capsid encoding region; (ii) the neomycin phosphotransferace gene (NPTII); (iii) the IRES from encephalomyocarditis virus (EMCV), inserted downstream of the NPTII gene and which directs translation of HCV proteins NS2 or NS3 to NS5B; and (iv) the 3′-UTR. After transfection of Huh-7 cells, only those cells supporting HCV RNA replication expressed the NPTII protein and developed resistance against the drug G418. While the cell lines derived from such G418 resistant colonies contained substantial levels of replicon RNAs and viral proteins, only 1 in 10⁶ transfected Huh-7 cells supported HCV replication.

Similar selectable HCV replicons were constructed based on an HCV-H genotype 1a infectious clone (Blight et al., Science 2000 290:1972-74). The HCV-H derived replicons were unable to establish efficient HCV replication, suggesting that the earlier-constructed replicons of Lohmann (1999), supra, were dependent on the particular genotype 1 b consensus cDNA clone used in those experiments. Blight et al. (2000), supra, reproduced the construction of the replicon made by Lohmann et al. (1999), supra, by carrying out a PCR— based gene assembly procedure and obtained G418-resistant Huh-7 cell colonies. Independent G418-resistant cell clones were sequenced to determine whether high-level HCV replication required adaptation of the replicon to the host cell. Multiple independent adaptive mutations that cluster in the HCV nonstructural protein NS5A were identified. The mutations conferred increased replicative ability in vitro, with transduction efficiency ranging from 0.2 to 10% of transfected cells as compared to earlier-constructed replicons in the art, e.g., the I₃₇₇/NS3-3′ replicon had a 0.0001% transduction efficiency.

Recently, Qi et al. described the construction of HCV genotype 1b replicon shuttle vectors that allow the cloning of patient-derived full-length NS3/4A gene in order to assess mutations that would confer resistance to candidate HCV protease inhibitors in development (Qi et al., Antiviral Res. 2009 81:166-173). However, the chimeric replicons that actually contained patient-derived full-length NS3/4A were unable to replicate in cell culture and replication was observed only when the laboratory-derived Con1 NS3 helicase domain was placed in the replicon.

### SUMMARY OF THE INVENTION

The present invention features the development of a novel HCV replicon shuttle vector in which unique restriction enzyme sites are introduced at the 5′ end of the NS3 gene and the 3′ end of the NS4A gene such that full length NS3/4A sequences derived from the samples of HCV-infected patients can be cloned in the shuttle vector and the resulting replicons be evaluated for replication fitness and susceptibility to HCV NS3 protease inhibitors and to HCV RNA helicase inhibitors. Since an individual HCV-infected patient typically contains a genetically diverse virus population due to the high error rate of the NS5B RNA polymerase, the use of the shuttle vector of the present invention would allow the characterization of specific patient-derived NS3/4A variants and the sensitivity or resistance of these variants to drug treatment.

Accordingly, the present invention provides an HCV replicon shuttle vector comprising an HCV polynucleotide sequence wherein the HCV polynucleotide sequence contains unique restriction enzyme sequences flanking the 5′ end of the NS3A gene and the 3′ end of the NS4A gene. In one embodiment of the invention, the HCV polynucleotide sequence comprises, in order, a unique restriction enzyme sequence placed between 15 nucleotides 5′ and 5 nucleotides 3′ from the 5′ end of a polynucleotide sequence encoding a NS3 protein; a polynucleotide sequence encoding the NS3 protein; a polynucleotide sequence encoding a NS4A protein; a unique restriction enzyme sequence placed between 5 nucleotides 5′ and 15 nucleotides 3′ from the 3′ end of a polynucleotide sequence encoding the NS4 protein; a polynucleotide sequence encoding a NS4B protein; a polynucleotide sequence encoding a NS5A protein; and a polynucleotide sequence encoding a NS5B protein.

In another embodiment of the invention, the unique restriction enzyme sequence at the 5′ end of the polynucleotide sequence encoding the NS3 protein recognizes AsiSI and the unique restriction enzyme sequence at the 3′ end of the polynucleotide sequence encoding the NS4A protein recognizes FspAI or FseI. In still another embodiment of the invention, the HCV replicon shuttle vector comprises an HCV polynucleotide sequence selected from SEQ ID NO:5 or SEQ ID NO:8.

A further embodiment of the present invention provides a method for assessing the effectiveness of an HCV NS3 protease inhibitor or an HCV RNA helicase inhibitor to control an HCV infection in a subject comprising the steps of providing a sample from the subject infected with HCV, PCR-amplifying polynucleotide sequences encoding the NS3 protein and the NS4A protein from a plurarity of HCV quasispecies present in the sample with the use of a sense-strand primer which comprises a unique restriction enzyme sequence, and an anti-sense strand primer which comprises a different unique restriction enzyme sequence, cloning said PCR-amplifed polynucleotide sequences into an HCV replicon shuttle vector to produce chimeric HCV replicon plasmids, linearizing said chimeric HCV replicon plasmids and subjecting said linearized plasmids to in vitro transcription to produce chimeric HCV replicon RNAs, and transfecting a Huh7 cell line with said HCV replicon RNAs and measuring replication level of said HCV replicon RNAs in the presence or absence of the HCV NS3 protease inhibitor or the HCV RNA helicase inhibitor.

A still further embodiment of the present invention provides a method for assessing the effectiveness of an HCV NS3 protease inhibitor or an HCV RNA helicase inhibitor to control an HCV infection in a subject comprising the steps of providing a sample from the subject infected with HCV, PCR-amplifying polynucleotide sequences encoding the NS3 protein and the NS4A protein from a plurarity of HCV quasispecies present in the sample with the use of a sense-strand primer which comprises a unique restriction enzyme sequence, and an anti-sense strand primer which comprises a different unique restriction enzyme sequence, cloning said PCR-amplifed polynucleotide sequences into an HCV replicon shuttle vector to produce chimeric HCV replicon plasmids, transforming said plasmids into cells to generate a plurarity of colonies of transformed cells, pooling said colonies and isolating chimeric HCV replicon plasmids from the pooled colonies, linearizing said chimeric HCV replicon plasmids, subjecting said linearized plasmids to in vitro transcription to produce chimeric HCV replicon RNAs, and transfecting Huh7 cell line with said HCV replicon RNAs and measuring replication level of said HCV replicon RNAs in the presence or absence of the HCV NS3 protease inhibitor or the HCV RNA helicase inhibitor.

The foregoing and other advantages and features of the invention, and the manner in which the same are accomplished, will become more readily apparent upon consideration of the following detailed description of the invention taken in conjunction with the accompanying examples, which illustrate exemplary embodiments.

### BRIEF DESCRIPTION OF THE DRAWINGS

FIG. 1 shows plasmid maps of the replicon shuttle vector, pSC_{—}1b_NS3/4A/lacZ_AsiSI_FseI (A) and pSC_{—}1b_NS3/4A/lacZ_AsiSI_FspAI (B).

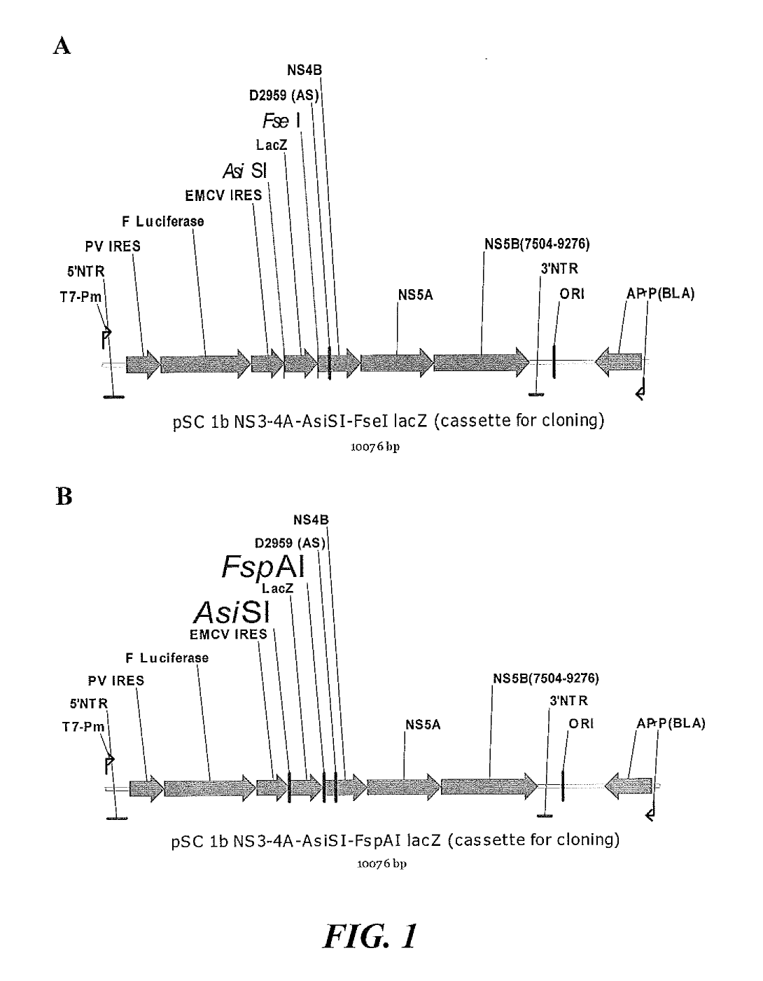

### DETAILED DESCRIPTION OF THE INVENTION

### Definitions

The term “HCV replicon” refers to a nucleic acid from the Hepatitis C virus that is capable of directing the generation of copies of itself. As used herein, the term “replicon” includes RNA as well as DNA, and hybrids thereof. For example, double-stranded DNA versions of HCV genomes can be used to generate a single-stranded RNA transcript that constitutes an HCV replicon. The HCV replicons can include full length HCV genome or HCV subgenomic contructs also referred as a “subgenomic replicon”. For example, the subgenomic replicons of HCV described herein contain most of the genes for the non-structural proteins of the virus, but are missing most of the genes coding for the structural proteins. Subgenomic replicons are capable of directing the expression of all of the viral genes necessary for the replication of the viral subgenome, replication of the sub-genomic replicon, without the production of viral particles.

A basic HCV replicon is a subgenomic construct containing an HCV 5′-untranslated (UTR) region, an HCV NS3-NS5B polyprotein encoding region, and a HCV 3′-UTR. Other nucleic acid regions can be present such as those providing for HCV NS2, structural HCV protein(s) and non-HCV sequences.

The HCV 5′-UTR region provides an internal ribosome entry site (IRES) for protein translation and elements needed for replication. The HCV 5′-UTR region includes naturally occurring HCV 5′-UTR extending about 36 nucleotides into a HCV core encoding region, and functional derivatives thereof. The 5′-UTR region can be present in different locations such as site downstream from a sequence encoding a selection protein, a reporter, protein, or an HCV polyprotein.

In addition to the HCV 5′-UTR-PC region, non-HCV IRES elements can also be present in the replicon. The non-HCV IRES elements can be present in different locations including immediately upstream the region encoding for an HCV polyprotein. Examples of non-HCV IRES elements that can be used are the EMCV IRES, poliovirus IRES, and bovine viral diarrhea virus IRES.

The HCV 3′-UTR assists HCV replication. HCV 3′ UTR includes naturally occurring HCV 3′-UTR and functional derivatives thereof. Naturally occurring 3′-UTRs include a poly U tract and an additional region of about 100 nucleotides.

The NS3-NS5B polyprotein encoding region provides for a polyprotein that can be processed in a cell into different proteins. Suitable NS3-NS5B polyprotein sequences that may be part of a replicon include those present in different HCV strains and functional equivalents thereof resulting in the processing of NS3-NS5B to produce a functional replication machinery. Proper processing can be measured for by assaying, for example, NS5B RNA dependent RNA polymerase.

A “vector” is a piece of DNA, such as a plasmid, phage or cosmid, to which another piece of DNA segment may be attached so as to bring about the replication, expression or integration of the attached DNA segment. A “shuttle vector” refers to a vector in which a DNA segment can be inserted into or excised from a vector at specific restriction enzyme sites. The segment of DNA that is inserted into shuttle vector generally encodes a polypeptide or RNA of interest and the restriction enzyme sites are designed to ensure insertion of the DNA segment in the proper reading frame for transcription and translation.

A variety of vectors can be used to express a nucleic acid molecule. Such vectors include chromosomal, episomal, and virus-derived vectors, e.g., vectors derived from bacterial plasmids, from bacteriophage, from yeast episomes, from yeast chromosomal elements, including yeast artificial chromosomes, from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., (1989) Molecular Cloning: A Laboratory Manual. 2nd edn. Cold Spring Harbor Laboratory Press, Cold Spring Harbor, N.Y., USA.

A vector containing the appropriate nucleic acid molecule can be introduced into an appropriate host cell for propagation or expression using known techniques. Host cells can include bacterial cells including, but not limited to, E. coli, Streptomyces, and Salmonella typhimurium, eukaryotic cells including, but not limited to, yeast, insect cells, such as Drosophila, animal cells, such as Huh-7, HeLa, COS, HEK 293, MT-2T, CEM-SS, and CHO cells, and plant cells.

Vectors generally include selectable markers that enable the selection of a subpopulation of cells that contain the recombinant vector constructs. The marker can be contained in the same vector that contains the nucleic acid molecules described herein or may be on a separate vector. Markers include tetracycline- or ampicillin-resistance genes for prokaryotic host cells and dihydrofolate reductase or neomycin resistance for eukaryotic host cells. However, any marker that provides selection for a phenotypic trait will be effective.

A “polynucleotide” or “nucleic acid molecule” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. “Polynucleotide” also embraces relatively short polynucleotides, often referred to as oligonucleotides.

In addition, the term “DNA molecule” refers only to the primary and secondary structure of the molecule, and does not limit it to any particular tertiary forms. Thus, the term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “RNA molecule” refers to the polymeric form of ribonucleotides in its either single-stranded form or a double-stranded helix form. In discussing the structure of particular RNA molecules, sequence may be described herein according to the normal convention of giving the sequence in the 5′ to 3′ direction.

The term “restriction enzyme sequence” refers to a specific double stranded-DNA sequence which is recognized and cut by bacterial enzymes, each of which cut double-stranded DNA at or near a specific nucleotide sequence. The restriction enzyme “AsiSI” recognizes the sequence

5′ GCGAT▾CGC 3′
3′ CGC▴TAGCG 5′

and cuts the double-stranded DNA at the indicated nucleotide position (shown with ^{▾}▴). The restriction enzyme “FspAI” recognizes the sequence

5′ RTGC▾GCAY 3′
3′ YACG▴CGTR 5′

where “R” represents “A” or “G” and “Y” represents “C” or “T” and cuts the. double-stranded DNA at the indicated nucleotide position.

The restriction enzyme “FseI” recognizes the sequence

5′ GGCCGG▾CC 3′
3′ CC▴GGCCGG 5′

and cuts the double-standed DNA at the indicated nucleotide position.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in PCR reactions, the primer is typically 15-25 nucleotides or longer in length. The primer must be of sufficient complementarity to the desired template to prime the synthesis of the desired extension product, i.e. to be able to anneal with the desired template strand in a manner sufficient to provide the 3′-hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence (e.g. a restriction enzyme recognition sequence) may be attached to the 5′-end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “chimeric” as used herein means a molecule of DNA that has resulted from DNA from two or more different sources that have been fused or spliced together.

As used herein, the term “quasispecies” means a collection of microvariants of a predominant HCV genome sequence (i.e. genotype), said microvariants being formed in a single infected subject or even in a single cell clone or even in a single cell clone as a result of high mutation rate during HCV replication.

The term “subject” as used herein refers to vertebrates, particular members of the mammalian species and includes, but not limited to, rodents, rabbits, shrews, and primates, the latter including humans.

The term “sample” refers to a sample of tissue or fluid isolated from a subject, including but not limited to, for example, plasma, serum, spinal fluid, lymph fluid, the external sections of the skin, respiratory, intestinal and genitourinary tracts, tears, saliva, milk, blood cells, tumors, organs, and also samples of in vitro cell culture constituents (including but not limited to, conditioned medium resulting from the growth of cultured cells, putatively viral infected cells, recombinant cells, and cell components).

A cell has been “transformed” or “transfected” by exogenous or heterologous DNA or RNA when such DNA or RNA has been introduced inside the cell. The transforming or transfecting DNA or RNA may or may not be integrated (covalently linked) into chromosomal DNA making up the genome of the cell. For example, in prokaryotes, yeast, and mammalian cells, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. In the case of an HCV replicon that transforms a mammalian cell as described in the present invention, the RNA molecule, e.g., an HCV RNA molecule, has the ability to replicate semi-autonomously. Huh-7 cells carrying the HCV replicons are detected either by the presence of a selection marker or a reporter gene present on the replicon.

A “clone” refers to a population of cells derived from a single cell or common ancestor generally by the process of mitosis.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Construction of Plasmids

The transient HCV GT-1b Con1 replicon vector (rep PI-luc/ET) was obtained from R. Bartenschlager. Briefly, it includes the poliovirus internal ribosome entry site (IRES), which controls the translation of the firefly luciferase gene. Downstream of the firefly luciferase gene, the IRES from the encephalomyocarditis virus (EMCV) controls the translation of the HCV non-structural genes (NS3, NS4A, NS4B, NS5A and NS3/4A). The repPI-luc/ET vector was modified to replace the pBR322 backbone with the pUC18 backbone to generate replicon pPI-luc/ET/SC. Replicon pPI-luc/ET/SC replicated with similar levels to rep PI-luc/ET as disclosed in US Patent Publication No. US2008/0026952 by Dietrich et al., which is incorporated by reference in full herein.

Mutations were introduced into pPI-luc/ET/SC in order to put in unique restriction enzyme sequences at the 5′ end of the NS3 gene and at the 3′ end of the NS4A gene by using the QuikChange site-directed mutagenesis kit following the manufacturer's instructions (Stratagene, La Jolla, Calif., USA). An AsiSI restriction enzyme sequence was introduced at the end of the IRES sequence, immediately upstream of the 5′ end of the NS3 gene by using the following primers:

Sense primer
(SEQ ID NO: 1)
5′-CGGGGACGTGGTTTTCCTTTGAAAAACGCGATCGCACCATGGC
GCCTATTACG-3′
Anti-sense primer
(SED ID NO: 2)
5′-CGTAATAGGCGCCATGGTGCGATCGCGTTTTTCAAAGGAAAAC
CACGTCCCCG-3′

(The initiation codon of the NS3 gene is in bold.)

To introduce a FseI restriction enzyme sequence at the 3′ end of the NS4A gene, the following primers were used:

Sense primer
(SEQ ID NO: 3)
5′-GATGAGATGGAAGGGCCG*GCCTCACACCTCCCTTACATCG-3′
Anti-sense primer
(SEQ ID NO: 4)
5′-CGATGTAAGGGAGGTGTGAGGC*CGGCCCTTCCATCTCATC-3′

(The asterisk represents the 3′ end position of the NS4A gene.) This resulted in the construction of the shuttle vector pSC_{—}1b_NS3/4A_AsiSI_FseI. Replication capacity of this shuttle vector was similar to that of pPI-luc/ET/SC.

Next, the entire NS3/4A gene sequence was replaced by the beta-galactosidase (lacZ) coding sequence from pUC19 (GenBank Accession Number M77789). AsiSI and FseI restriction enzyme sequences were introduced at the 5′ end and at the 3′ end of the lacZ gene, respectively, by PCR amplification and resulted in generating the shuttle vector, pSC_{—}1b_NS3/4A/lacZ_AsiSI_FseI (SEQ ID NO:5).

An alternate NS3/4A shuttle vector with a FspAI restriction enzyme sequence replacing the FseI restriction enzyme sequence at the 3′ end of the NS4A gene was generated using pSC_{—}1b_NS3/4A/lacZ_AsiSI_FseI as the template and the following primers:

Sense primer
(SEQ ID NO: 6)
5′-GTCTCCGGGAGCTGGGTGC*GCATCACACCTCCCTTACA-3′
Anti-sense primer
(SEQ ID NO: 7)
5′-TGTAAGGGAGGTGTGATGC*GCACCCAGCTCCCGGAGAC-3′

(The asterisk represents the 3′ end position of the NS4A gene.) This resulted in the generation of the shuttle vector, pSC_{—}1b_NS3/4A/lacZ_AsiSI_FspAI (SEQ ID NO:8).

### Example 2

### Cloning of the NS3/4A PCR Samples Amplified from Infected Patients into the NS3/4A Replicon Shuttle Vectors

DNA sequences encoding the NS3/4A protein were generated from plasma obtained from patients infected with HCV using reverse transcription of RNA followed by PCRamplification of the reverse-transcribed product. Reverse transcription of RNA was performed using Taqman Reverse Transcription Reagents (Applied Biosystems, Foster City, Calif., USA) using the primer, 5′-ACCAGGTCCTCSGTGGAGG-3′ (SEQ ID NO:9) and according to the manufacturers' protocol. The synthesized cDNA was then PCR-amplified with the GC RICH PCR system (Roche Applied Science) to ensure high fidelity and robust yields. Annealing temperatures in the range of 50-52° C. were used depending on patient sample and primer combinations. Two PCR reactions with two independent primer sets used for the first round PCR were as follows:

The first set of primers used for the first round PCR were as follows:

| Sense primer | |
|---|---|
| 5′-GAGACCAAGATCATCACC-3′ | (SEQ ID NO: 10) |
| Anti-sense primer | |
| 5′-CCACCACGGGAGCAGC-3′ | (SEQ ID NO: 11) |

The second set of primers used for the first round PCR were as follows:

| Sense primer | |
|---|---|
| 5′-GGCAGACACCGCGGCGTGTGG-3′ | (SEQ ID NO: 12) |
| Anti-sense primer | |
| 5′-TTCCACATGTGCTTCGCCC-3′ | (SEQ ID NO: 13) |

The first round PCR products were then subject to a second PCR using primers that introduced an AsiSI restriction enzyme sequence at the 5′ end of the NS3 gene and either an FseI or FspAI restriction enzyme sequence at the 3′ end of the NS4A gene. The sense primer used to introduce the AsiSI restriction enzyme sequence was 5′-CTGTCTGTCTGCGATCGCACCATGGCGCCTATTAC GGCCTACTC-3′ (SEQ ID NO:14). The anti-sense primer used to introduce an FspAI restriction enzyme sequence was 5′-AGGGAGGTGTGATGCGCACTCTTCCATCTC-3′ (SEQ ID NO:15). The anti-sense primer used to introduce an FseI restriction enzyme sequence was 5′-CGCACTCTTCCATCTCATCGAACTC-3′ (SEQ ID NO:16)

Patient NS3/4A PCR amplicons were then purified using Qiagen PCR purification Columns, digested with the restriction endonuclease AsiSI and re-purified. The FspAI containing amplicon was then digested with FspAI. The final product was gel purified using Qiagen's Gel Extraction Kit.

The shuttle replicon pSC_{—}1b_NS3-4A-AsiSI-FseI_lacZ or pSC_{—}1b_NS3-4A-AsiSI-FspAI_lacZ the were prepared by digestion with restriction endonucleases AsiSI and FseI (New England Biolabs, Ipswitch, Mass., USA) or FspAI (Fermentas, Burlington, Ontario, Canada). The FseI vector was further treated with Klenow (New England Biolabs). The vectors were separated from digested insert by 1% agarose gel electrophoresis and the vector was gel purified using Qiagen's Gel Extraction Kit. Purified vectors were then treated with Shrimp Alkaline Phosphatase (Roche Applied Science).

Twenty-five ng of shuttle vector were ligated with the digested patient amplicons using T4 DNA ligase (Roche Applied Science) overnight at 14-16° C. Vector to insert ratio of 1:2 to 1:4 were routinely used. After overnight ligation, 5 ul of the reaction were transformed into 100 ul of One Shot OmniMAX 2 T Phage-Resistant Cells (Invitrogen, Carlsbad, Calif., USA) and plated after 1 hour of shaking at 3° C.

Ninety-six individual colonies were picked to inoculate 1200 ul of Terrific Broth (TB) supplemented with 50 μg/ml carbenecillin. This 96-well block was incubated overnight at 3° C. with shaking. The next day, 100 μl of each culture was combined and DNA was extracted by miniprep to represent the 96-clone pool. The remaining cultures were centrifuged and used for plasmid DNA extraction using Qiaprep 96 Turbo Mini-DNA Kit (Qiagen). These individual molecular clones represent the individual 96 variants used for the Replicon Phenotypic Assay were stored for future use.

Heterogeneous 96-clone pool plasmid DNA was submitted for sequencing to confirm the identity of patient samples and to screen for potential contaminating DNA prior to the in vitro transcription reaction. Fifty to one-hundred nanograms of plasmid DNA and 4 μmol of sequencing primers were routinely used.

### Example 3

### Replicon Phenotypic Assay

A. Preparation of In vitro Transcribed RNA

Five micrograms of DNA was linearized by Sca I restriction enzyme (Roche Applied Science). After overnight digestion at 37° C., the DNA was purified using Qiagen PCR purification kit. Eight microliters of linearized DNA was used for the in vitro transcription using T7 RiboMAX Express Large Scale RNA Production System kit following manufacturer's protocol (Promega, Madison, Wis., USA). After 2 hours of incubation at 37° C., DNase treatment was performed for 20 minutes at 37° C. to remove the DNA template. In vitro transcribed RNA was then purified using RNeasy mini kit following manufacturer's protocol (Qiagen).

### B. Hepatoma Cell Line Huh7

Cured hepatoma cell line Huh7 was obtained from R. Bartenschlager. Cells were cultured at 37° C. in a humidified atmosphere with 5% CO₂ in Dulbecco's Modified Eagle Medium (DMEM) supplemented with Glutamax and 100 mg/ml sodium pyruvate (Cat# 10569-010). The medium was further supplemented with 10% (v/v) FBS (Cat# 16000-036) and 1% (v/v) penicillin/streptomycin (Cat# 15140-122). All reagents were from Invitrogen.

### C. Determination of Transient Replicons Replication Level

Four million cured Huh7 cells were transfected with 10 μg of in vitro transcribed RNA using electroporation. Cells were resuspended in 12 ml of DMEM containing 5% Fetal Bovine Serum and plated in 96-well plate at 28,800 cells/well (in 90 μl final volume). Firefly luciferase reporter signal was read 96 hours using the Luciferase Assay system (Promega, cat #E1501).

### D. IC₅₀ Determination Using the Transient Replicon Assay

Four million cured Huh7 cells were transfected with 10 μg of in vitro transcribed RNA using electroporation. Cells were then resuspended in 12 ml of DMEM containing 5% FBS and plated in 96-well plate at 28,800 cells/well (in 90 μl final volume) Inhibitors were added 24 hours post-transfection in 3 fold dilutions at a final DMSO concentration of 1% and firefly luciferase reporter signal was read 72 hours after addition of inhibitors using the Luciferase Assay system (Promega, cat #E1501). The IC₅₀ values were assessed as the inhibitor concentration at which a 50% reduction in the level of firefly luciferase reporter was observed as compared to the level of firefly luciferase signal without the addition of compounds.

The replication capacities of pSC_{—}1b_NS3/4A/lacZ_AsiSI_FspAI shuttle vectors containing the NS3/4A gene from various patient samples were tested with the luciferase signal as the readout and compared to the replication capacity of the parent replicon, pPI-luc/ET/SC. The results are shown on Table 1. Table 1 also shows the inhibitory effects of the HCV protease inhibitor VX-950 on these replicons.

TABLE 1

| | Average | Average | Average Replication | IC₅₀ |
|---|---|---|---|---|
| Replicon/ | 4 hour RLU | 96 hour RLU | Capacity | VX-950 |
| Isolate | (n = 2) | (n = 2) | (96 hRLU/4 hRLU) | (μM) |
| pPI- | 208,162 | 3,251,777 | 15.62 | 0.253 |
| luc/ET/SC | | | | |
| 52620 | 243,933 | 18,735 | 0.076 | 0.092 |
| 135641 | 469,324 | 1,185,002 | 2.567 | 0.175 |
| 66200 | 271,568 | 3,391,970 | 12.564 | 0.332 |
| 61793 | 293,801 | 802,771 | 2.809 | 0.381 |
| 98 | 279,608 | 157,827 | 0.567 | 0.205 |
| 73837-2 | 96,054 | 1,287,394 | 12.486 | 0.228 |
| 34 | 690,322 | 126,847 | 0.170 | 0.268 |
| 135635 | 209,118 | 691,696 | 3.243 | 0.394 |
| 5903 | 177,789 | 63,602 | 0.348 | 0.097 |
| 135640 | 131,656 | 205,948 | 1.393 | 0.385 |
| 135649 | 536,817 | 1,753,944 | 3.269 | 0.108 |
| 34026 | 787,764 | 1,898,966 | 2.436 | 0.048 |
| RO256 | 684,419 | 270,146 | 0.405 | 0.093 |

RLU represents level of firefly luciferase signal observed after 4 hours or 96 hours following transfection

1. An HCV replicon shuttle vector comprising an HCV polynucleotide sequence that comprises, in order:
(a) a unique restriction enzyme sequence placed between 15 nucleotides 5′ and 5 nucleotides 3′ from the 5′ end of a polynucleotide sequence encoding a NS3 protein;
(b) a polynucleotide sequence encoding the NS3 protein;
(c) a polynucleotide sequence encoding a NS4A protein;
(d) a unique restriction enzyme sequence placed between 5 nucleotides 5′ and 15 nucleotides 3′ from the 3′ end of a polynucleotide sequence encoding the NS4A protein;
(e) a polynucleotide sequence encoding a NS4B protein;
(f) a polynucleotide sequence encoding a NS5A protein; and
(g) a polynucleotide sequence encoding a NS5B protein.

2. The HCV replicon shuttle vector of claim 1 wherein the polynucleotide sequence encoding the NS3 protein, the NS4A protein, or both the NS3 protein and the NS4A protein has been modified or deleted such that the protease activity of the NS3 protein is non-functional.

3. The HCV replicon shuttle vector of claim 1 or claim 2 wherein the unique restriction enzyme sequence at the 5′ end of the polynucleotide sequence encoding the NS3 protein recognizes AsiSI and the unique restriction enzyme sequence at the 3′ end of the polynucleotide sequence encoding the NS4A protein recognizes FspAI or FseI.

4. An HCV replicon shuttle vector comprising an HCV polynucleotide sequence selected from SEQ ID NO:5 or SEQ ID NO:8.

5. A method for assessing the effectiveness of an HCV NS3 protease inhibitor or an HCV RNA helicase inhibitor to control an HCV infection in a subject comprising the steps of:
(a) providing a sample from the subject infected with HCV;
(b) PCR-amplifying polynucleotide sequences encoding a NS3 protein and a NS4A protein from a plurarity of HCV quasispecies present in the sample with the use of a sense-strand primer which comprises a unique restriction enzyme sequence, and an anti-sense strand primer which comprises a different unique restriction enzyme sequence;
(c) cloning said PCR-amplifed polynucleotide sequences into an HCV replicon shuttle vector to produce chimeric HCV replicon plasmids;
(d) linearizing said chimeric HCV replicon plasmids and subjecting said linearized plasmids to in vitro transcription to produce chimeric HCV replicon RNAs; and
(e) transfecting a Huh7 cell line with said HCV replicon RNAs and measuring replication level of said HCV replicon RNAs in the presence or absence of the HCV NS3 protease inhibitor or the HCV RNA helicase inhibitor.

6. The method of claim 5 wherein the HCV replicon shuttle vector of step (c) comprises the HCV replicon shuttle vector of claim 1.

7. The method of claim 5 wherein the HCV replicon shuttle vector of step (c) comprises the HCV replicon shuttle vector of claim 2.

8. The method of claim 5 wherein the HCV replicon shuttle vector of step (c) comprises the HCV replicon shuttle vector of claim 3.

9. The method of claim 5 wherein the HCV replicon shuttle vector of step (c) comprises the HCV replicon shuttle vector of claim 4.

10. A method for assessing the effectiveness of an HCV NS3 protease inhibitor or an HCV RNA helicase inhibitor to control an HCV infection in a subject comprising the steps of:
(a) providing a sample from the subject infected with HCV;
(b) PCR-amplifying polynucleotide sequences encoding a NS3 protein and a NS4A protein from a plurarity of HCV quasispecies present in the sample with the use of a sense-strand primer which comprises a restriction enzyme sequence that recognizes AsiSI, and an anti-sense strand primer which comprises a restriction enzyme sequence that recognizes FspAI or FseI;
(c) cloning said PCR-amplifed polynucleotide sequences into an HCV replicon shuttle vector to produce chimeric HCV replicon plasmids;
(d) linearizing said chimeric HCV replicon plasmids and subjecting said linearized plasmids to in vitro transcription to produce chimeric HCV replicon RNAs; and
(e) transfecting a Huh7 cell line with said HCV replicon RNAs and measuring replication level of said HCV replicon RNAs in the presence or absence of the HCV NS3 protease inhibitor or the HCV RNA helicase inhibitor.

11. The method of claim 10 wherein the HCV replicon shuttle vector of step (c) comprises the HCV replicon shuttle vector of claim 1.

12. The method of claim 10 wherein the HCV replicon shuttle vector of step (c) comprises the HCV replicon shuttle vector of claim 2.

13. The method of claim 5 wherein the HCV replicon shuttle vector of step (c) comprises the HCV replicon shuttle vector of claim 3.

14. The method of claim 5 wherein the HCV replicon shuttle vector of step (c) comprises the HCV replicon shuttle vector of claim 4.

15. A method for assessing the effectiveness of an HCV NS3 protease inhibitor or an HCV RNA helicase inhibitor to control an HCV infection in a subject comprising the steps of:
(a) providing a sample from the subject infected with HCV;
(b) PCR-amplifying polynucleotide sequences encoding a NS3 protein and a NS4A protein from a plurarity of HCV quasispecies present in the sample with the use of a sense-strand primer comprising nucleotide sequence SEQ ID NO: 14, and an anti-sense strand primer comprising a nucleotide selected from SEQ ID NO:15 or SEQ ID NO:16;
(c) cloning said PCR-amplifed polynucleotide sequences into an HCV replicon shuttle vector to produce chimeric HCV replicon plasmids;
(d) linearizing said chimeric HCV replicon plasmids and subjecting said linearized plasmids to in vitro transcription to produce chimeric HCV replicon RNAs; and
(e) transfecting Huh7 cell line with said HCV replicon RNAs and measuring replication level of said HCV replicon RNAs in the presence or absence of the HCV NS3 protease inhibitor or the HCV RNA helicase inhibitor.

16. The method of claim 15 wherein the HCV replicon shuttle vector of step (c) comprises the HCV replicon shuttle vector of claim 1.

17. The method of claim 15 wherein the HCV replicon shuttle vector of step (c) comprises the HCV replicon shuttle vector of claim 2.

18. The method of claim 15 wherein the HCV replicon shuttle vector of step (c) comprises the HCV replicon shuttle vector of claim 3.

19. The method of claim 15 wherein the HCV replicon shuttle vector of step (c) comprises the HCV replicon shuttle vector of claim 4.

20. A method for assessing the effectiveness of an HCV NS3 protease inhibitor or an HCV RNA helicase inhibitor to control an HCV infection in a subject comprising the steps of:
(a) providing a sample from the subject infected with HCV;
(b) PCR-amplifying polynucleotide sequences encoding a NS3 protein and a NS4A protein from a plurarity of HCV quasispecies present in the sample with the use of a sense-strand primer which comprises a unique restriction enzyme sequence, and an anti-sense strand primer which comprises a different unique restriction enzyme sequence;
(c) cloning said PCR-amplifed polynucleotide sequences into an HCV replicon shuttle vector to produce chimeric HCV replicon plasmids;
(d) transforming said plasmids into cells to generate a plurarity of colonies of transformed cells;
(e) pooling said colonies and isolating chimeric HCV replicon plasmids from the pooled colonies;
(f) linearizing said chimeric HCV replicon plasmids from step (e) and subjecting said linearized plasmids to in vitro transcription to produce chimeric HCV replicon RNAs; and
(g) transfecting Huh7 cell line with said HCV replicon RNAs and measuring replication level of said HCV replicon RNAs in the presence or absence of the HCV NS3 protease inhibitor or the HCV RNA helicase inhibitor.

21. The method of claim 20 wherein the HCV replicon shuttle vector of step (c) comprises the HCV replicon shuttle vector of claim 1.

22. The method of claim 20 wherein the HCV replicon shuttle vector of step (c) comprises the HCV replicon shuttle vector of claim 2.

23. The method of claim 20 wherein the HCV replicon shuttle vector of step (c) comprises the HCV replicon shuttle vector of claim 3.

24. The method of claim 20 wherein the HCV replicon shuttle vector of step (c) comprises the HCV replicon shuttle vector of claim 4.

25. A method for assessing the effectiveness of an HCV NS3 protease inhibitor or an HCV RNA helicase inhibitor to control an HCV infection in a subject comprising the steps of:
(a) providing a sample from the subject infected with HCV;
(b) PCR-amplifying polynucleotide sequences encoding a NS3 protein a NS4A protein from a plurarity of HCV quasispecies present in the sample with the use of a sense-strand primer which comprises a restriction enzyme sequence that recognizes AsiSI, and an anti-sense strand primer which comprises a restriction enzyme sequence that recognizes FspAI or FseI;
(c) cloning said PCR-amplifed polynucleotide sequences into an HCV replicon shuttle vector to produce chimeric HCV replicon plasmids;
(d) transforming said plasmids into cells to generate a plurarity of colonies of transformed cells;
(e) pooling said colonies and isolating chimeric HCV replicon plasmids from the pooled colonies;
(f) linearizing said chimeric HCV replicon plasmids from step (e) and subjecting said linearized plasmids to in vitro transcription to produce chimeric HCV replicon RNAs; and
(g) transfecting Huh7 cell line with said HCV replicon RNAs and measuring replication level of said HCV replicon RNAs in the presence or absence of the HCV NS3 protease inhibitor or the HCV RNA helicase inhibitor.

26. The method of claim 25 wherein the HCV replicon shuttle vector of step (c) comprises the HCV replicon shuttle vector of claim 1.

27. The method of claim 25 wherein the HCV replicon shuttle vector of step (c) comprises the HCV replicon shuttle vector of claim 2.

28. The method of claim 25 wherein the HCV replicon shuttle vector of step (c) comprises the HCV replicon shuttle vector of claim 3.

29. The method of claim 25 wherein the HCV replicon shuttle vector of step (c) comprises the HCV replicon shuttle vector of claim 4.

30. A method for assessing the effectiveness of an HCV NS3 protease inhibitor or HCV RNA helicase inhibitor to control an HCV infection in a subject comprising the steps of:
(a) providing a sample from the subject infected with HCV;
(b) PCR-amplifying polynucleotide sequences encoding a NS3 protein and a NS4A protein from a plurarity of HCV quasispecies present in the sample with the use of a sense-strand primer comprising nucleotide sequence SEQ ID NO: 14, and an anti-sense strand primer comprising a nucleotide selected from SEQ ID NO:15 or SEQ ID NO:16;
(c) cloning said PCR-amplifed polynucleotide sequences into an HCV replicon shuttle vector to produce chimeric HCV replicon plasmids;
(g) transforming said plasmids into cells to generate a plurarity of colonies of transformed cells;
(h) pooling said colonies and isolating chimeric HCV replicon plasmids from the pooled colonies;
(i) linearizing said chimeric HCV replicon plasmids from step (e) and subjecting said linearized plasmids to in vitro transcription to produce chimeric HCV replicon RNAs; and
(g) transfecting Huh7 cell line with said HCV replicon RNAs and measuring replication level of said HCV replicon RNAs in the presence or absence of the HCV NS3 protease inhibitor or the HCV RNA helicase inhibitor.

31. The method of claim 30 wherein the HCV replicon shuttle vector of step (c) comprises the HCV replicon shuttle vector of claim 1.

32. The method of claim 30 wherein the HCV replicon shuttle vector of step (c) comprises the HCV replicon shuttle vector of claim 2.

33. The method of claim 30 wherein the HCV replicon shuttle vector of step (c) comprises the HCV replicon shuttle vector of claim 3.

34. The method of claim 30 wherein the HCV replicon shuttle vector of step (c) comprises the HCV replicon shuttle vector of claim 4.
